(19) United States
(12) Patent Application Publication
(10) Pub. No.: US 2024/0090760 A1
(43) Pub. Date: Mar. 21, 2024

(54) METHODS AND SYSTEMS FOR DETECTING VASCULATURE

(71) Applicants: Singapore Health Services Pte Ltd, Singapore (SG); Nanyang Technological University, Singapore (SG)

(72) Inventors: Leopold SCHMETTERER, Singapore (SG); Wing Kee Damon WONG, Singapore (SG); Ai Ping YOW, Singapore (SG)

(21) Appl. No.: 18/262,003

(22) PCT Filed: Jan. 19, 2022
(86) PCT No.: PCT/SG2022/050018

§ 371 Date: Jul. 18, 2023

(30) Foreign Application Priority Data
Jan. 19, 2021 (SG) 10202100558X

(51) Int. Cl. A61B 3/00; A61B 3/10; A61B 3/12; G06T 7/00; G06T 7/11; G06V 10/25; G16H 50/20

(52) U.S. Cl. CPC A61B 3/0025; A61B 3/102; A61B 3/1233; G06T 7/0012; G06T 7/11; G06V 10/25; G16H 50/20; G06T 2207/10101; G06T 2207/20084; G06T 2207/30041; G06T 2207/30101; G06V 2201/03

(57) ABSTRACT

The invention relates to a system of detecting vasculature in optical coherence tomography (OCT) image data of a tissue of a subject, the OCT image data comprising OCT scan data and OCT angiography (OCTA) scan data, the system comprises segmenting the OCT scan data to locate a layer of interest in the tissue; generating an en face vascular network map from the OCTA scan data; projecting one or more vascular regions from the en face vascular network map onto the layer of interest in a cross-sectional image of the OCT scan data to define one or more regions of interest (ROIs), wherein respective ROIs are defined by the intersection between the vascular regions and the layer of interest; and identifying vascular objects in the one or more ROIs. In the preferred embodiment, the tissue is retina, vessels are removed from the layer of interest and the retinal nerve fibre layer (RNFL) thickness is determined.

### TECHNICAL FIELD

The present disclosure relates generally to methods and systems for detecting vasculature, for example in retinal image data obtained via Optical Coherence Tomography.

### BACKGROUND

Glaucoma, the world's major cause of irreversible blindness in adults, is an eye disease characterized by the progressive degeneration of retinal ganglion cells (RGCs) and their axons. RGCs are essential for vision and the loss of RGCs leads to structural changes in the optic nerve head and thinning of the peripapillary retinal nerve fibre layer (RNFL) associated with gradual visual field loss.

Patients with early-stage glaucoma may be unaware of visual field loss until later stages of the disease where the RGCs have been permanently damaged and adversely affected the vision.

RNFL thinning can be indicative of RGC loss in glaucoma and studies have shown the potentiality of RNFL thickness measurement for early detection and monitoring of glaucoma progression using optical coherence tomography (OCT). OCT is an interferometric technique enabling in vivo imaging of retinal structures containing both neuronal and vascular components. RNFL thickness measurements include both neuronal and vascular components. However, the inclusion of vascular components can potentially affect thickness measurements and image processing operations for the assessment of glaucoma.

The vascular component can be visualised and quantified non-invasively using OCT angiography (OCT-A). Vascular assessment forms an important part of the detection and assessment of the progression of glaucoma. In particular, the peripapillary vessel density in glaucomatous eyes could be lower compared to those in normal eyes, and a strong correlation with the visual field and disease severity. Furthermore, the reduced density of peripapillary capillaries could be significantly associated with increased visual field severity in advanced primary open angle glaucoma (POAG) eyes.

Optical Coherence Tomography (OCT) imaging enables clinicians to perform in vivo assessments of the underlying structure of the eye or other biological tissue to detect pathological changes. It also allows quantitative evaluation between baseline and follow-up scans to monitor disease progression and determine suitable interventions. When such measurements are taken, the OCT scans are segmented and the thickness of certain layers is quantified. These layers consist of different components including neurons, vessels, glial cells and other structures. The interest is, however, usually directed towards the number of neural cells, which is a biomarker of neuronal death in diseases such as glaucoma, diabetic retinopathy or other neurodegenerative diseases of the brain such as Alzheimer's disease.

Conventional quantitative measurements do not account for vasculature which affects the variability and diagnostic accuracy of tissue measurements. The influence of blood vessels in structural thickness measurements can significantly affect the variability of thickness profiles. Hence, there remains a need to account for vasculature in the tissues when performing structural measurements, for example when performing structural measurements of the retina for patients with ocular conditions.

Conventional approaches for detecting and segmenting vessels in OCT include the application of adaptive binarization method on cross-sectional OCT scans, the creation of shadowgraphs in cross-sectional OCT scans to assign the lateral vessel positions followed by the use of the active shape model method to segment the vessels, and the detection of vessel axial margins in cross-sectional OCT scans by tracking the optical shadows cast onto the outer retina and manually adjusting the incorrect axial margins. However, these conventional approaches have only been applied to cross-sectional OCT scans and have largely relied on the presence of optical shadow artefacts for vessel localization. As such, only larger vessels with salient shadows are likely to be detected while smaller vessels with less prominent shadows can be under-detected, leading to an under-representation of the vascular components.

It would be desirable to overcome or alleviate at least one of the above-described problems, or at least to provide a useful alternative system or method for detecting vasculature.

### SUMMARY

In a first aspect, the disclosure provides a method of detecting vasculature in OCT image data of a tissue of a subject, the OCT image data comprising optical coherence tomography (OCT) scan data and OCT angiography (OCTA) scan data, the method comprising:
segmenting the OCT scan data to locate a layer of interest in the tissue;
generating an en face vascular network map from the OCTA scan data;
projecting one or more vascular regions from the en face vascular network map onto the layer of interest in a cross-sectional image of the OCT scan data to define one or more regions of interest (ROIs), wherein respective ROIs are defined by the intersection between the vascular regions and the layer of interest; and
identifying vascular objects in the one or more ROIs.

In some embodiments, the tissue is a retina of the subject.

The vascular objects may be identified by: shape fitting within the ROI; a Hough transform; or a Watershed transform.

Some embodiments of the method comprise removing the vascular objects from the layer of interest to generate an image of one or more non-vascular components of the layer of interest. The one or more non-vascular components may comprise a neuronal component.

In some embodiments of the method, said segmenting is carried out using a convolutional neural network. The convolutional neural network may be U-Net.

Some embodiments of the method comprise determining one or more clinical parameters based on the vascular objects and/or the image of the one or more non-vascular components.

In some embodiments, the one or more vascular regions in the en face vascular map reside in a circumpapillary region. The one or more clinical parameters may comprise circumpapillary retinal nerve fibre layer (RNFL) thickness.

In some embodiments, the layer of interest is selected according to a disease model.

In a second aspect, the disclosure provides a system for detecting vasculature in OCT image data of a tissue of a subject, the OCT image data comprising optical coherence tomography (OCT) scan data and OCT angiography (OCTA) scan data, the system comprising at least one processor in communication with machine-readable storage having stored thereon instructions for causing the at least one processor to carry out a method as disclosed herein.

In a third aspect, the disclosure provides non-transitory computer-readable storage having stored thereon processor-executable instructions for causing at least one processor to carry out a method as disclosed herein.

In a fourth aspects, the disclosure provides a system for detecting vasculature in OCT image data of a tissue of a subject, the system comprising: at least one processor (processors(s)); a memory accessible to the processor, the memory comprising program code executable by the processors(s) to:
receive OCT image data comprising optical coherence tomography (OCT) scan data and OCT angiography (OCTA) scan data;
segment the OCT scan data to locate a layer of interest in the tissue;
generate an en face vascular network map from the OCTA scan data;
project one or more vascular regions from the en face vascular network map onto the layer of interest in a cross-sectional image of the OCT scan data to define one or more regions of interest (ROIs), wherein respective ROIs are defined by the intersection between the vascular regions and the layer of interest; and
identify vascular objects in the one or more ROIs.

### BRIEF DESCRIPTION OF THE DRAWINGS

Some embodiments of a system and method for detecting vasculature in retinal image data, in accordance with present teachings will now be described, by way of non-limiting example only, with reference to the accompanying drawings in which:

FIG. 1 shows a flowchart of a method for detecting vasculature;

FIG. 2 is an overview of a framework for detecting vascular components in OCT data;

FIG. 3 shows images from en face OCT (1st row) and OCTA (2nd row) with corresponding en face vessel maps (right);

FIG. 4 is an illustration of lateral vascular localisation using an en face OCTA vascular network map (top) and the corresponding circumpapillary OCTA image (bottom);

FIG. 5 illustrates segregation of neuronal-vascular components in the layer of interest;

FIG. 6 shows (a) a single vessel mask V (circle) is generated based on half the lateral extent (r) and the determined centroid of the region (Cx, Cy); (b) a circular vessel mask generated by fitting a circular model for each vessel ROI; (c) a binarized RNFL segmentation obtained using a trained U-Net model; and (d) a binarized RNFL image with vessels excluded by masking the RNFL segmentation with the circular vessel masks;

FIG. 7 shows an example of a specific application of the disclosed approach in the analysis of an OCT-based image for glaucoma diagnosis;

FIG. 8(a) shows a vessel-removed RNFL image generated using Otsu thresholding;

FIG. 8(b) shows a segmented RNFL image from an OCTA circumpapillary scan, and a histogram of pixel values therefrom;

FIG. 8(c) shows a vessel-removed RNFL image using a cutoff defined by the histogram of FIG. 8(b);

FIG. 9 shows a comparison of ROC curves between circumpapillary thickness measurement with and without vessels removal for glaucoma diagnosis;

FIG. 10 shows a system for detecting vasculature;

FIG. 11 shows another example of a specific application of the disclosed approach in the analysis of an OCT, OCTA based image for glaucoma diagnosis; and

FIG. 12 a Receiver operating characteristic (ROC) curves for glaucoma detection.

### DETAILED DESCRIPTION

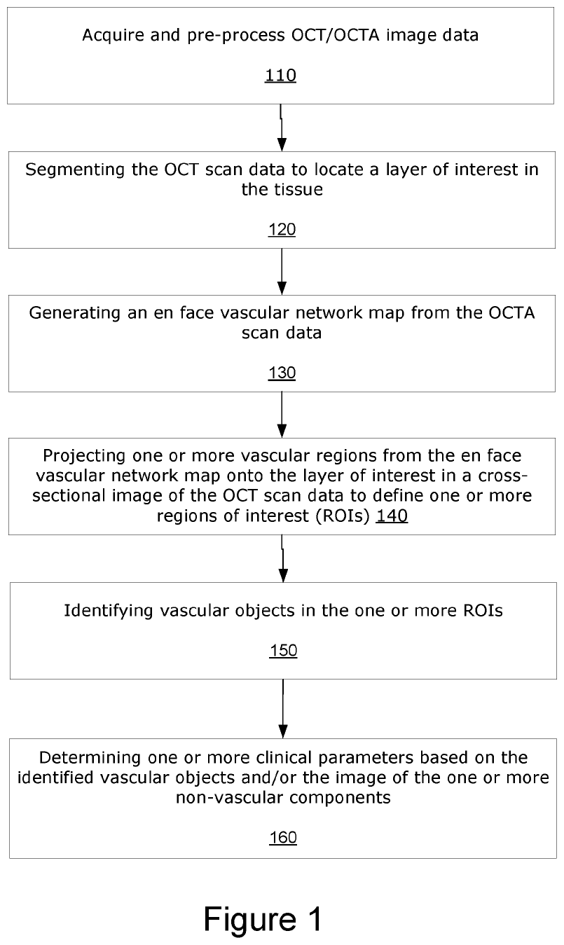

Described below is a framework to localise the vessels within a layer of interest in an OCT scan and to differentiate the neuronal and vascular components for further analyses and modelling. Embodiments may comprise three main processes: acquisition and pre-processing of OCT/OCTA images; depth-resolved vascular localisation; and segregation of neuronal-vascular components. FIG. 1 shows a flowchart of a process of segregating vessels from the nerve fibre layer executable by system 1000 of FIG. 10. The system 1000 comprises at least one processor 1010 in communication with a memory/storage 1030. The memory 1030 comprises OCT image data 210 and program code (NuVAS program code 1032) to process the OCT image data 210 and detect vasculature by executing the steps illustrated in the method of the flowchart of FIG. 1.

Quantitative structural measurements from OCT data include the influence of vasculature which could potentially affect the variability of measurements and can confound the monitoring and identification of disease in patients. OCT Angiography (OCTA) technology enables the detailed visualisation of vasculature which could aid in the detection and quantification of vascular changes in patients with ocular disease.

The disclosed framework combines vasculature information from OCTA and structural data from OCT to detect and remove the influence of vascular structures, generating a better measure of tissue changes in OCT for higher diagnostic accuracy. This is important to ensure that changes in thickness measurements are attributable to pathological changes and are not confounded by the presence of vessels.

Embodiments of the present disclosure have one or more of the following features and/or advantages:
Fully-automated approach for obtaining vessel-removed structural measurements
In situ localisation of volumetric blood vessels using a multi-view technique that combines different views to optimize the detection of vessel structures
Vessel-removed measurements enable better measurement of the tissue of interest by removing the confounding effect of vascular structures.

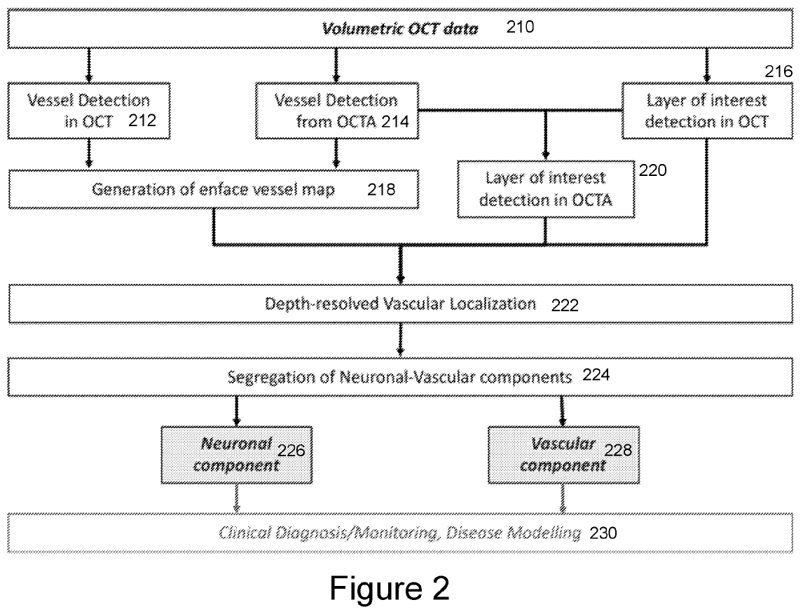

Some embodiments of the present disclosure relate to a framework to differentiate the vascular and neuronal components in OCT and OCTA. The disclosed framework is referred to herein as Neuronal-VAscular Separator (NuVAS). The flowcharts of FIG. 1 and FIG. 2 exemplify the various steps of the NuVAS framework. NuVAS incorporates deep learning methods and biologically-inspired image processing to automatically determine the contributions of blood vessels in tissue layers (including retinal tissue layers) and adjust clinically-relevant metrics to account for these contributions. The flow of the NuVAS framework is presented below in FIG. 2. This approach provides improved diagnostic performance for the detection of neurodegenerative diseases of the eye and the brain. Separating neuronal and vascular components enables the improvement of complex modelling approaches such as deep learning techniques for the prediction of neurogenerative and systemic diseases.

At step 110 of FIG. 1, OCT image data comprising optical coherence tomography (OCT) scan data and OCT angiography (OCTA) scan data 210 is received or acquired by the system 1000. As illustrated in FIG. 2, before segregating the vascular and neuronal components, the volumetric OCT data 210 is used to generate an en face vessel map 218 and detect a layer of interest at step 120. The volumetric OCT information comprises a set of A-scans (depth-wise information on refractive index changes) regarding the tissue of a subject. The en face vessel map contains vascular information which can be generated automatically by projecting the vessels vertically from the volumetric OCT data.

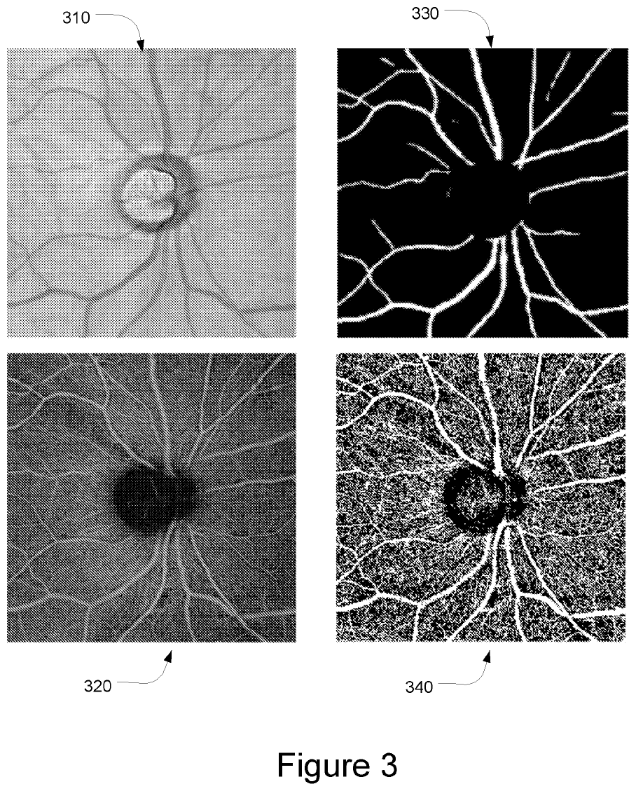

Another approach for generating the en face vessel map is by employing signal amplitude decorrelation between consecutive transverse cross-sectional OCT scans acquired at the same retinal location (214 in FIG. 2 and step 130 of FIG. 1). Such a method is also known as OCT angiography (OCTA). Vascular flow in blood vessels leads to higher decorrelation, while static tissue leads to lower decorrelation values. This difference in correlation values enables the generation of an OCT angiogram showing the vascular network in the retina. The detected vascular network may then be binarized using image processing techniques such as adaptive thresholding to extract the vascular information and to generate the en face vessel maps. FIG. 3 shows an example of an OCT (image 310) and OCTA (image 320) image of the vascular network at the superficial layer, with the corresponding generated en face vessel maps (image 330 being a map based on image 310 and image 340 being a map based on image 320).

To isolate the neuronal component (inclusive of vessels) in a specific region axially, identification of the layer of interest depending on the disease model is required. Layer detection (216 and 220 in FIG. 2 and steps 120 of FIG. 1) involves the process of clustering an OCT image into several coherent sub-regions according to the extracted features. The layers then can be automatically segmented from volumetric OCT data using image processing techniques or advanced techniques such as convolutional networks. U-Net is one neural network architecture that can be adopted to solve biomedical image segmentation problems. The network merges a convolutional network architecture with a deconvolutional architecture to output the semantic segmentation of various layers. Model training is performed with the manual demarcation of layers as the ground truth. After obtaining the segmented layer of interest from OCT, the OCTA signals are masked by the obtained segmentation to isolate the vascular component in the layer of interest.

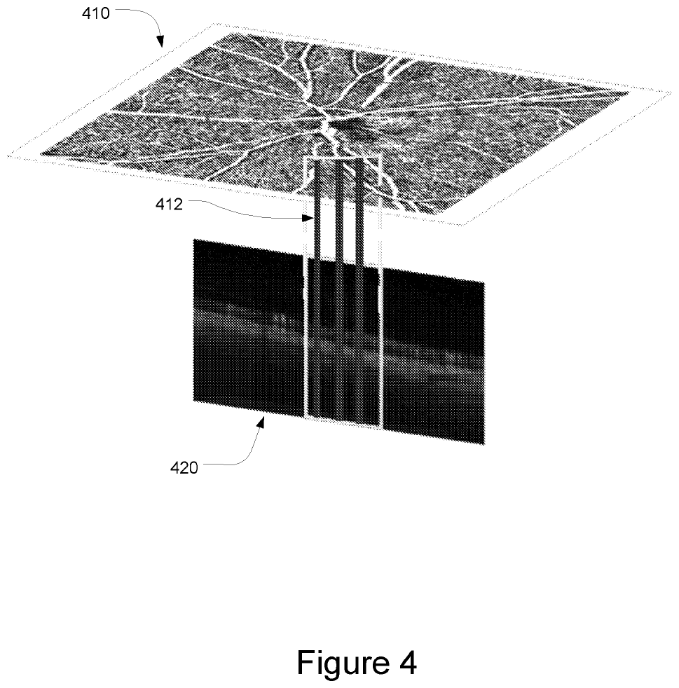

Thereafter, the vessels in the specific region which are extracted from the en face vessel map are then vertically projected onto the cross-sectional images and combined with the segmented layer of interest to demarcate individual blood vessels within the layer at step 140 of FIG. 1. FIG. 4 illustrates the vascular localization method using the en face OCTA vessel map and a corresponding circumpapillary protocol. The circle in image 410 represents the circular scan protocol. Vessels detected from the OCTA map are projected onto the circumpapillary using vertical bars 412 on the OCTA image 420 which defines the lateral extent of the vessels.

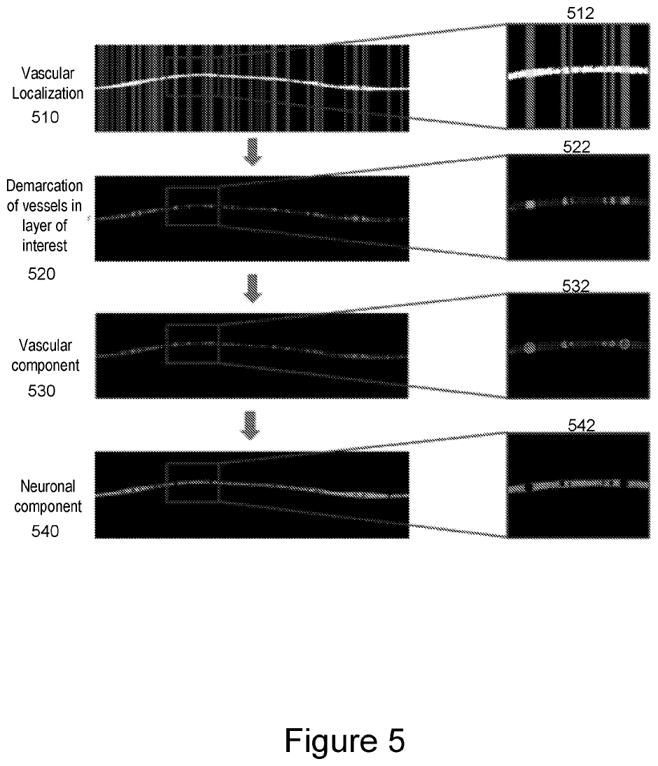

In one example, as illustrated in FIG. 5, regions of interest indicating the presence of vascular structures from the map (vascular localization map 510) are vertically projected onto the corresponding locations on the OCTA cross-sectional image 520 and combined with the detected retinal layer segmentation for vascular localization. This is referred to as shape fitting within the ROI. Specifically, the lateral extent of the signals for each vessel in the OCTA cross-sectional image is determined from the vertical vascular map projections, whereas the axial extent was bounded by the upper and lower limits of the segmented retinal layer. Using these extents, the ROIs containing each vessel was constrained laterally and axially. A circular model is adopted for the vessel shape. Depth-resolved localization of the vessel in each ROI was determined from the centroid of the constrained OCTA signals, while the vessel-calibre was estimated from lateral extents of the vertical projections from the vascular network map. Using these parameters, circular vascular models were fitted for each ROI. Other shapes may also be adopted. Alternatively, other image processing techniques such as based on the Hough Transform or the Watershed Transform may also be used. Lastly, the fitted vessels are segregated from the segmented retina to segregate the vascular components (images 530, 532) from the neuronal components (image 540, 542), from which measurements and parameters for disease diagnosis and modelling are generated as part of step 160 of the flowchart of FIG. 1.

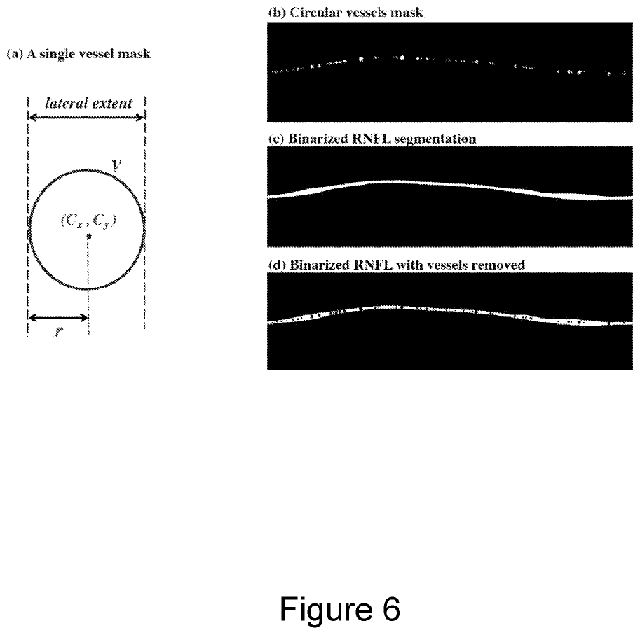

Further details of depth-resolved vascular localisation are described with reference to FIGS. 6(a) to 6(d).

As illustrated in FIG. 6(a), the centroid (Cₓ, C_{y}) of each 8-connected vessel region is automatically determined based on the vascular localization as exemplified in images 510 and 520. The disclosed embodiments operate under the assumption that the circumpapillary scan is perpendicular to the vessels and the segmented RNFL contains the cross-section of the entire vessel. Circular vessel masks V are created using the circle equation:

Vᵢ=[Mₕ−C_{y,i}]²+[M_{w}−C_{x,i}]²<r² (1)

where i is the i^{th} vessel region, r is the radius of the circular vessels which is defined by half the lateral extent, and Mₕ and M_{w} refer to the height and width of the mesh grid generated based on the circumpapillary OCT scan. An example of the generated circular vessel masks is shown in FIG. 6(b).

Thereafter, these generated circular vascular masks are excluded from the neuronal layer as shown in FIG. 6(d). The two structural parameters, namely the RNFL thickness RT and vessel-to-thickness VT ratio are computed using the neuronal layer data of FIG. 6(d) to compare the correlation with age. The RNFL thickness including the vascular components along the circular scan is averaged across all A-scans, to obtain an average RNFL thickness value RTₐᵥₑ for each eye:

$$R {T}_{ave} = \frac{1}{N} \times {\sum }_{n = 1}^{N} {t}_{n} \qquad ( 2 )$$

where N is the total number of columns in binarized RNFL segmentation as shown in FIG. 6(c) and tₙ refers to the thickness of the segmented RNFL at the n^{th} A-scan. The thickness of the RNFL excluding the vascular component was also computed using (2) and denoted as RT_{ave,nv} for later analysis. Additionally, the embodiments compute the RNFL thickness after excluding the major vessels which were only visible in OCT scans (RT_{ave,nm}) as a further comparison. The major vessels are manually selected from the detected vessel regions based on the OCT scans.

Subsequently, the embodiments calculate the proportion of vessels relative to the RNFL cross-sectional area VT ratio for all eyes. This parameter is defined as the ratio between total vessel area and the RNFL area before excluding vessels, which was computed as follows:

$$VT = \frac{{\sum }_{t = 1}^{I} {V}_{i}}{{R}_{area}} \qquad ( 3 )$$

where Vᵢ is a circular vessel mask with values calculated using (1), I is the total number of circular vessel masks within the RNFL and Rₐᵣₑₐ is the area of RNFL before excluding vessels which was computed using (4).

Rₐᵣₑₐ=wΣᵢ₌₁^{I}RTᵢ (4)

where w is the width of the circumpapillary cross-sectional scan and RTᵢ is the RNFL thickness at each A-scan.

After segregation, both vascular and neuronal components can potentially be used for clinical diagnosis as well as for disease monitoring and treatment. Vascular components are important in the diagnosis of retinal diseases such as diabetic retinopathy and glaucoma, while neuronal components enable clinicians to identify structural changes in disease progression and provide early intervention. Further post-processing of these extracted components can be performed to obtain quantitative and objective metrics such as vessel density, vessel size and structural thickness of retinal layers, which can improve clinical diagnosis. Besides generating useful clinical parameters, the two separated components can also be used as higher-level features to construct new learning features for ocular and neurodegenerative disease modelling. Both neuronal and vascular components can be input as two separate layers to a deep convolutional neural network, to model the disease progression and predict the risk in individuals.

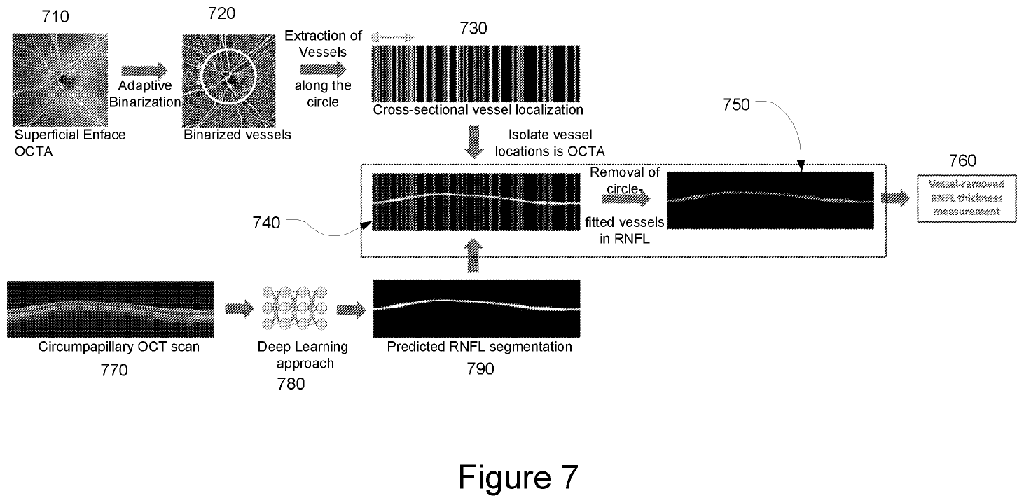

A study was performed in accordance with an embodiment of the present disclosure for generating vessel-removed retinal nerve fibre layer (RNFL) thickness for glaucoma progression monitoring. Some embodiments of the present disclosure can be applied in the context of glaucoma diagnosis, specifically as applied on the circumpapillary retinal nerve fibre layer (RNFL) thickness measurements. Glaucoma is a progressive optic neuropathy that leads to loss of retinal ganglion cells and thinning of RNFL. Circumpapillary RNFL thickness measurements, which is defined as the circular region around the optic nerve head, have been used for glaucoma diagnosis and monitoring. However, conventional measurements do not discriminate between nerve fibre axons and retinal vasculature. The disclosed embodiments enable the exclusion of the influence of vasculature when measuring the RNFL thickness, particularly in measuring axon loss in glaucoma. FIG. 7 shows the process flow diagram of a process for obtaining vessel-removed RNFL thickness measurement using an embodiment of the present disclosure.

The circumpapillary RNFL may be extracted from an optic disc-centered volumetric OCT scan as follows. The volumetric data (data of image 720) is first vertically projected (images 730 and 740) to generate a two-dimensional en face view where the boundary of the optic disc was defined with the optic disc centre determined automatically.

A circumpapillary cross-sectional scan (image 770) of diameter 3.46 mm centered at the optic disc was then extracted from the volume. To reduce signal noise and improve the visibility of RNFL boundaries, the generated circumpapillary scan was averaged with two additional circumpapillary scans at diameters 3.44 mm and 3.46 mm. The RNFL layer was automatically segmented from the resulting averaged image using the U-Net based convolutional neural network without applying filters or pre-processing techniques. The advantage of using a U-Net based network is that it merges a convolutional network architecture with a deconvolutional architecture to output the semantic segmentation of layer of interest, allowing extraction of a vast number of features without losing the spatial information when the resolution decreases. The trained model takes in an input image of a cross-sectional OCT scan (image 770) which was first resized to 512×512 and generates a binarized circumpapillary RNFL segmentation (image 790) which was used to mask the cross-sectional OCTA scan.

The corresponding en face OCTA image 710 was binarized by applying adaptive thresholding to extract the vascular information and generate the en face vessel map of image 720. After which, the vessels around the circular scan (a distance of 3.46 mm to the centre of the optic disc) were extracted and vertically projected onto a cross-sectional plane illustrated in image 730. It was then combined with the segmented circumpapillary RNFL to demarcate individual blood vessels within the layer. After which, the vessel-removed RNFL (image 759) is obtained by removing the circle-fitted vessels from the layer. The thickness of the vessel-removed RNFL was measured for evaluating the diagnostic performance which will be discussed next.

The diagnostic accuracy of the proposed NuVAS approach of the disclosure was evaluated and compared other methods for generating a vessel-removed RNFL profile using a dataset of 343 eyes which were imaged using the Plex Elite 9000 OCT system (Carl Zeiss Meditec, USA) with a wavelength of 1050 nm, a scanning rate of 100,000 A-scans/s and 6 mm×6 mm imaging protocol, centred at the optic disc. Of the 343 eyes in the dataset, 250 were clinically diagnosed glaucomatous eyes and 93 were healthy eyes.

Two alternative methods for vessel extraction were also evaluated, and are illustrated as follows:

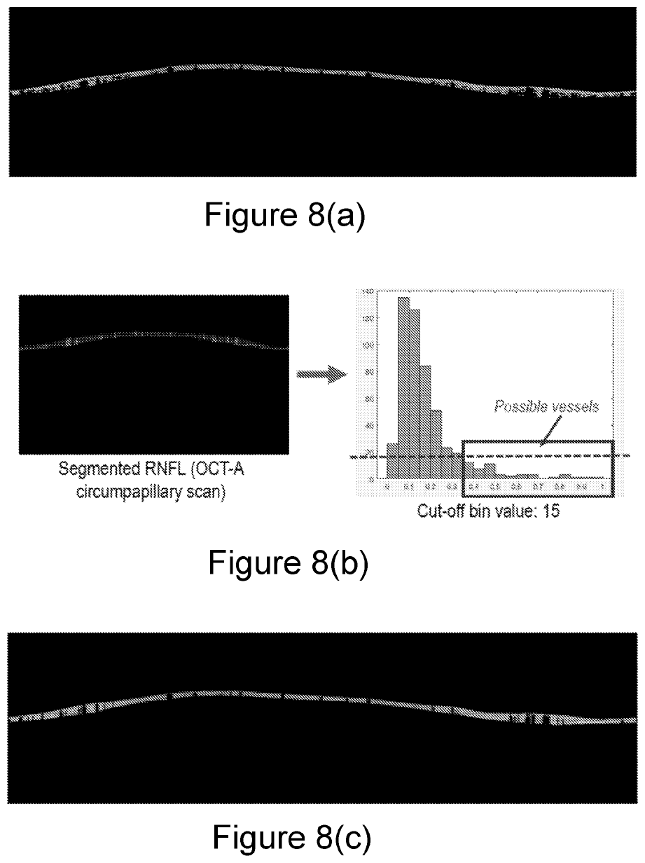

1. Otsu: Vessels were detected by applying Otsu thresholding on the OCTA signal data and then removed from OCT segmented circumpapillary RNFL. The resulting vessel-removed RNFL image is shown in FIG. 8(a).

2. Histogram-based: Vessels were detected based on the histogram of the pixel intensity value in the OCTA cross-sectional circumpapillary scan. In OCTA, vessel pixels have higher intensity due to the higher decorrelation values. Based on the distribution of intensity range in the image, an optimal threshold (i.e. histogram bin of 15 and below, FIG. 8(b)) was empirically selected to distinguish vessels within RNFL. These detected structures were then excluded from the segmented circumpapillary RNFL to generate a vessel-removed RNFL image as shown in FIG. 8(c).

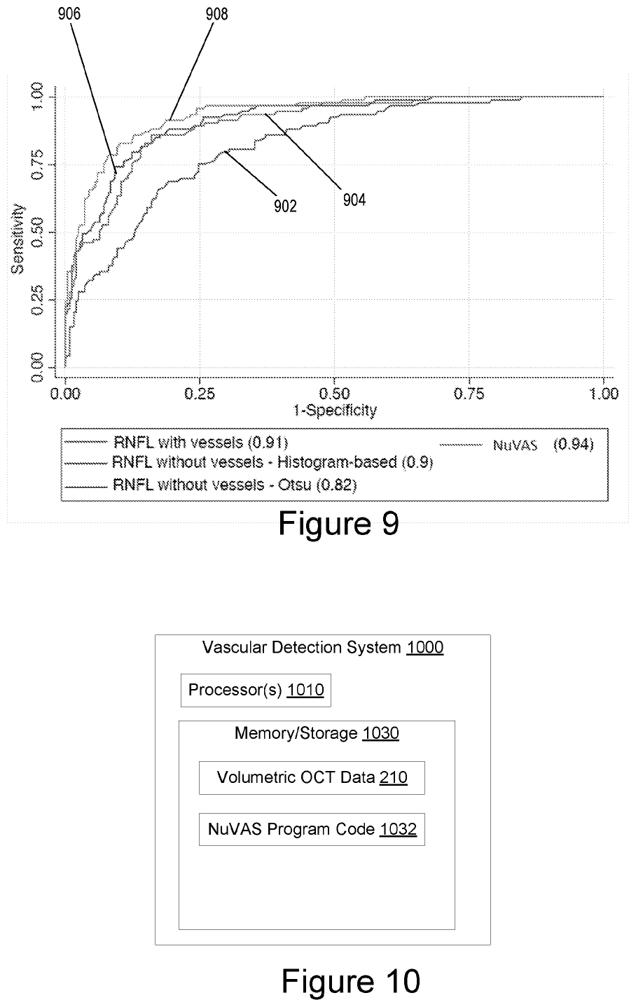

The diagnostic performance of these different methods was assessed using Receiver Operating Characteristic (ROC) curve analysis and compared using the Area under the ROC (AUC) metric. FIG. 9 shows the ROC curves of the circumpapillary RNFL thickness measurement with and without vessel removal in distinguishing between glaucomatous and non-glaucomatous eyes. The result shows the diagnostic accuracy for the standard clinical measure of RNFL (curve 906) is AUC 0.91. By using an approach according to the present disclosure, the vessel-removed RNFL obtained a diagnostic accuracy of AUC 0.94 (curve 908) and is the highest compared to the two alternative methods (curve 902 for Otsu vessel extraction and curve 904 for Histogram-based vessel extraction). As such, the method of the present disclosure shows improved diagnostic performance and could potentially better aid clinicians in detecting and monitoring glaucoma progression.

The conventional clinical way of disease monitoring is to perform direct measurement of structural changes in the retina without accounting for the presence of blood vessels. The presence of blood vessels affects the accuracy of clinical assessment. The disclosed NuVAS framework has the following advantages over conventional clinical practice:
Improved diagnostic accuracy in identifying the early stage of ocular conditions without the influence of blood vessels on structural changes
Reduced variability in structural changes for better ocular disease progression monitoring
Extracted volumetric vascular information which can be used for diagnostic purposes in both ocular and systemic conditions

Study Using NuVAS

Study Population

A cross-sectional study comprising both healthy subjects and subjects with POAG was performed from July 2018 to June 2019 to evaluate the effectiveness of the disclosed systems and methods.

Clinical Examination

All participants underwent a comprehensive eye examination, including assessment of best-corrected visual acuity using a logarithm of the minimum angle of resolution chart (LogMAR chart, The Lighthouse, NY), autorefractometry, intraocular pressure measurement using Goldman applanation tonometry, OCT and OCT-A imaging. Pupils were dilated with a drop of tropicamide 1% (Gutt Mydriacyl) drops prior to imaging. Visual fields were assessed using standard automated perimetry using 24-2 Swedish Interactive Threshold Algorithm (Humphrey visual field analyzer Carl Zeiss Meditec, Inc, Dublin, CA). The visual field test was considered reliable if fixation losses were less than 33%, and false-positive and false-negative errors were less than 20%.

POAG eyes were defined based on clinical diagnosis, which included the presence of glaucomatous optic neuropathy (defined as loss of neuroretinal rim with a vertical cup-to-disc ratio of >0.7 or an inter-eye asymmetry of >0.2 and/or notching attributable to glaucoma) with compatible visual field loss, open angles on gonioscopy, glaucoma hemifield test outside normal limits and absence of secondary causes of glaucomatous optic neuropathy.

OCT/OCT-A Image Acquisition and Scanning Protocol

OCT and OCT-A images were obtained using a commercial swept-source OCT (SS-OCT) system (PLEX Elite 9000, Carl Zeiss Meditec, Inc., Dublin, CA, USA) with a tunable centre wavelength of 1050 nm and a scanning rate of 100 kHz. Each eye underwent a 6×6 mm field of view imaging protocol centered at the optic nerve head. Each acquired volumetric scan was composed of 500 cross-sectional images with each image consisting of 500 A-scans. The depth-resolved angiographic signals were obtained to form OCT-A images using an optical microangiography (OMAG) technique. Image quality was manually assessed by trained graders. Poor quality images with signal strength less than 6, severe motion or shadow artefacts were excluded from the analysis.

Peripapillary RNFL Segmentation

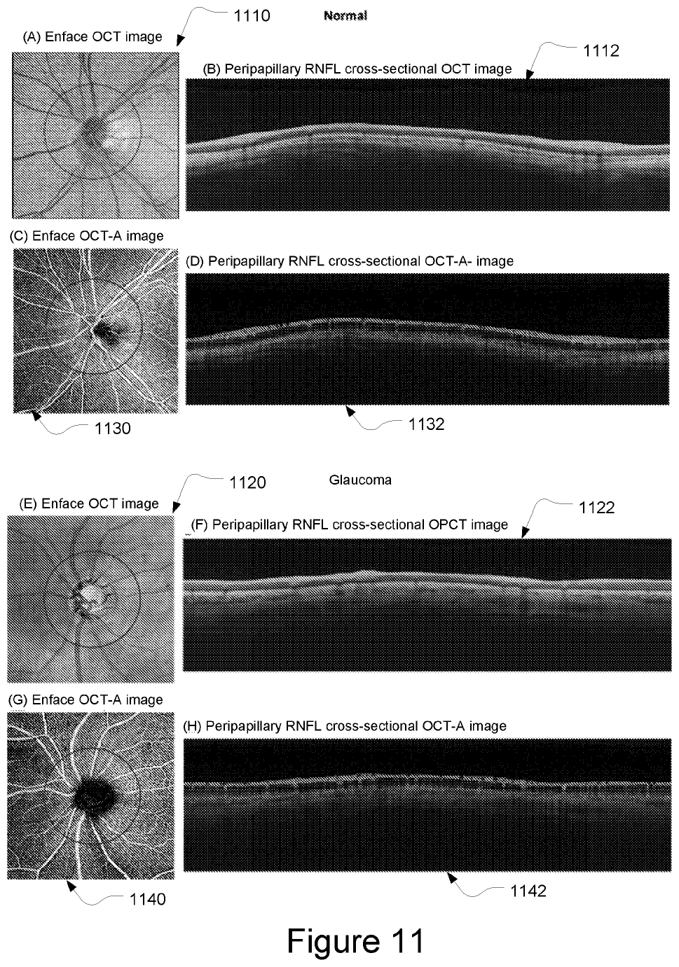

The acquired OCT scans were exported to MATLAB (Mathworks Inc. Natick, MA, USA) and reconstructed into three-dimensional OCT volumes. Enface projections of these volumes were used to delineate the optic disc boundaries and automatically determine the centre of the optic nerve head (ONH) (FIG. 11, images 1110, 1120). With the centre of ONH, the embodiments generated the peripapillary RNFL cross-sectional image for each acquired OCT scan (FIG. 11, images 1112, 1122) and performed automated segmentation of peripapillary RNFL using the U-Net21 based convolutional neural network. For each segmented peripapillary RNFL, the embodiments computed the average RNFL thickness (RNFLT) thickness metric.

Vascular and Neuronal Component in Peripapillary RNFL The superficial capillary plexus (SCP) which is defined by the inner limiting membrane (ILM) and inner plexiform layer (IPL) was obtained from a review software, PLEX Elite 9000 Review Software (version 1.6, Carl Zeiss Meditec, Dublin, CA, USA). Enface OCT-A images were then generated from the maximum projection of the SCP and for further extraction of vascular components (FIG. 11, images 1130 and 1140). The embodiments applied adaptive thresholding on the enface OCT-A images to binarize the vascular information.

The vascular structures along a circular scan were extracted from the binarized vasculature map and vertically projected onto the corresponding locations on the peripapillary RNFL cross-sectional image. Finally, the vertically projected vascular cross-sectional map was combined with the peripapillary RNFL segmentation to localize individual vessels. Large vessels were selected based on the visibility in OCT scans and capillaries were the remaining vascular structures in OCT-A scans after exclusion of the large vessels.

The RNFL mainly consists of RGC axons which are ensheathed by glial cells and blood vessels. After excluding the detected vascular components (including larger vessels and capillaries), the remaining segmented peripapillary RNFL is referred to as the neuronal component (FIG. 11, images 1132 and 1142). An additional two thickness metrics were computed: the average RNFL thickness excluding large vessels (LVRT; large vessels-removed RNFL thickness), and the average RNFL thickness excluding all vessels (AVRT; all vessels-removed RNFL thickness). In addition, three vascular metrics were computed in the peripapillary RNFL: the total area of the large vessels (TLVA; total large vascular area), the total capillary area (TCA; total capillaries area) and total area for all vessels (TVA; total vascular area).

Statistical Analysis

Descriptive statistics included mean and standard deviation for normally distributed variables. Independent-sample t tests were used to compare the differences in age, intra-ocular pressure (TOP), spherical equivalent (SE), visual field mean deviation (MD), and OCT signal strength between normal and glaucomatous eyes. χ2 test was used for categorical variables. Pearson's correlation analysis was carried out to evaluate the associations between the computed metrics (RNFLT, LVRT, AVRT, TLVA, TCA and TVA) and clinical variables. The computed metrics were included in a logistic regression analysis to assess the effect of the vascular component on the diagnostic performance in glaucomatous eyes. The study assessed the performance of RNFLT, LVRT, AVRT, AVRT+TLAV, AVRT+TCA and AVRT+TLVA+TCA in discriminating normal and glaucomatous eyes using the receiver-operating characteristic (ROC) curve and compared the areas under the ROC curves (AUCs) using a method proposed by DeLong et al. ‘ Comparing the areas under two or more correlated receiver operating characteristic curves: a nonparametric approach. Biometrics. 1988; 44(3):837-845.’ To avoid bias due to inter-eye correlations from each participant, 95% CIs for the AUC were calculated using a non-parametric bootstrapping resampling procedure (N=1000 samples), with each participant acting as the unit of resampling. All statistical analyses were performed with the commercial statistical software, Stata version 16.0 (StataCorp). P values less than 0.05 were considered to be statistically significant.

Sturdy Results

A total of 325 eyes of 213 participants who met the inclusion and exclusion criteria were included in this analysis. Of which the 325 eyes, there were 75 eyes of 43 control individuals and 250 eyes of 170 patients with POAG. Demographic characteristics of the study participants are presented in Table 1. The mean age of the normal and glaucoma groups was 56.1±14.2 years and 64.0±12.7 years, respectively. Patients with glaucoma had an average visual field mean deviation of −3.65±3.14 dB. There were significant differences in age, intra-ocular pressure (TOP) and OCT signal strength between the normal and glaucoma groups (P<0.001). There was no difference in spherical equivalent (SE) (P=0.284), systolic blood pressure (P=0.756) and diastolic blood pressure (P=0.202) between both groups.

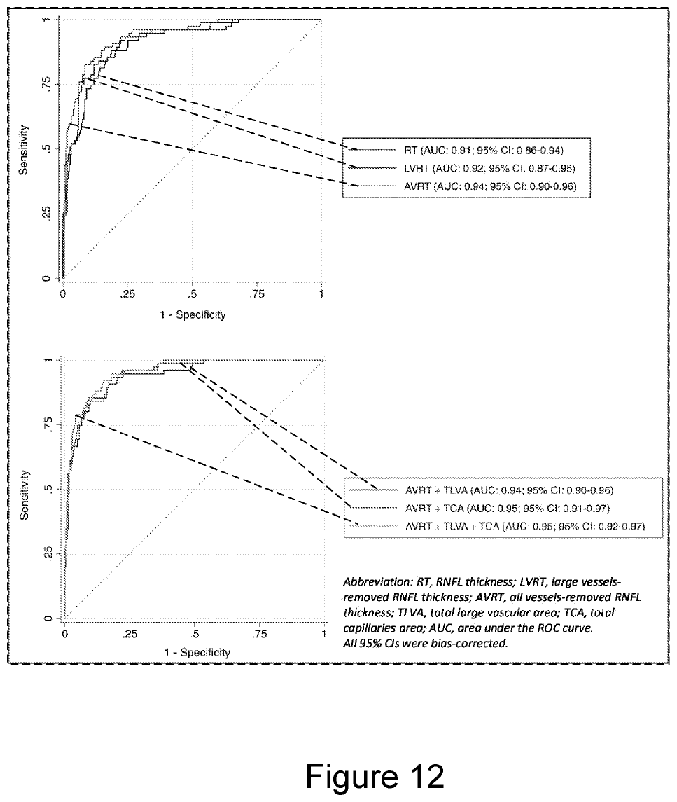

RNFLT, LVRT, AVRT, TLVA, TCA and TVA were computed, and the distributions of these metrics are illustrated in FIG. 12. Pearson's correlation analysis was performed to determine the associations between biometric variables (age, systolic blood pressure, diastolic blood pressure and IOP) and the computed metrics (RNFLT, LVRT, AVRT, TLVA, TCA and TVA) in both normal and glaucoma groups. Table 2 presents the correlations for each metric in both groups. In the normal group, the reduced average RNFL thickness with all vessels removed (AVRT) was shown to be significantly correlated with increasing age (r=−0.383, P<0.001). TCA had a positive correlation with increasing age (r=0.394, P<0.001) and higher systolic blood pressure (r=0.277, P=0.016) for normal eyes. There was no significant correlation between the other four computed metrics (RNFLT, LVRT, TLVA and TVA) and the biometric variables. In glaucomatous eyes, the average RNFL thickness (r=−0.200, P=0.002), average RNFL thickness with large vessels removed (r=−0.255, P<0.001) and average RNFL thickness with all vessels removed (r=−0.256, P<0.001) were shown to be correlated with increasing age. The two metrics for total vascular area of large vessels (TLVA) and all vessels (TVA) were not significantly correlated with all biometric variables. TCA showed a positive correlation with increasing IOP in glaucomatous eyes (r=0.136, P=0.032).

The study evaluated the correlations between all the computed metrics and visual field mean deviation in glaucomatous eyes. All five metrics were correlated with increasing severity of visual loss, with the average RNFL thickness with all vessels removed (AVRT) having the highest correlation (r=0.319, P<0.001). Total vascular area including both large vessels and capillaries (TVA) also had a moderate correlation with visual field mean deviation (r=0.204, P=0.001). There was no significant correlation between TCA and visual field loss (r=0.003, P=0.967).

TABLE 1
Demographic Characteristics, Biometric Variables and
OCT Signal Strength of the normal and glaucoma groups

| | Study Group | | | | |
|---|---|---|---|---|---|
| Demographics | Normal | | Glaucoma | | P value^{a} |
| Participants, n | 43 | | 170 | | NA |
| Gender, n | | | | | |
| Men | 9 | | 117 | | <.001 |
| Women | 34 | | 53 | | |
| Ethnicity, n | | | | | |
| Chinese | 36 | | 153 | | 0.087 |
| Malay | 4 | | 6 | | |
| Indian | 2 | | 10 | | |
| Others | 1 | | 1 | | |
| Eyes, n | 75 | | 250 | | NA |
| Age, mean (SD), y | 56.1 | (14.2) | 64.0 | (12.7) | <.001 |
| Spherical | −1.49 | (3.35) | −1.94 | (2.98) | 0.284 |
| equivalent, | | | | | |
| mean (SD), D | | | | | |
| Visual field | NA | | −3.65 | (3.14) | NA |
| mean deviation, | | | | | |
| mean (SD), dB | | | | | |
| Intra-ocular pressure, | 16.55 | (3.08) | 14.00 | (2.85) | <.001 |
| mean (SD), mm Hg | | | | | |
| Blood pressure, | | | | | |
| mean (SD), mm Hg | | | | | |
| Systolic | 139.06 | (20.92) | 138.24 | (19.60) | 0.756 |
| Diastolic | 76.84 | (8.15) | 75.30 | (9.42) | 0.202 |
| OCT signal strength, | 9.25 | (0.64) | 8.80 | (0.97) | <.001 |
| mean (SD) | | | | | |

Abbreviation:
NA, not applicable;
SD, standard deviation
^{a}P values were obtained with independent-sample t test for continuous variables and with χ² tests for categorical variables.

TABLE 2
Summary of Pearson Correlations Between Computed
Metrics, Biometric Variables and OCT Signal
Strength in Normal and Glaucomatous eyes

| Computed | Correlation | Normal | | Glaucoma | |
|---|---|---|---|---|---|
| Metrics | Variable | Pearson, r | P value | Pearson, r | P value |
| RNFLT | Age | −0.383 | <.001^{a} | −0.200 | 0.002^{a} |
| | Systolic blood | −0.196 | 0.092 | −0.159 | 0.012^{a} |
| | pressure | | | | |
| | Diastolic blood | −0.002 | 0.984 | −0.000 | 0.999 |
| | pressure | | | | |
| | Intra-ocular | 0.032 | 0.784 | 0.019 | 0.767 |
| | pressure | | | | |
| | Visual field mean | NA | NA | 0.297 | <.001^{a} |
| | deviation | | | | |
| | OCT signal | 0.427 | <.001^{a} | 0.273 | <.001^{a} |
| | strength | | | | |
| LVRT | Age | −0.359 | 0.002^{a} | −0.255 | <.001^{a} |
| | Systolic blood | −0.180 | 0.122 | −0.136 | 0.032^{a} |
| | pressure | | | | |
| | Diastolic blood | 0.001 | 0.991 | −0.001 | 0.990 |
| | pressure | | | | |
| | Intra-ocular | 0.045 | 0.703 | 0.088 | 0.164 |
| | pressure | | | | |
| | Visual field mean | NA | NA | 0.316 | <.001^{a} |
| | deviation | | | | |
| | OCT signal | 0.406 | <.001^{a} | 0.266 | <.001^{a} |
| | strength | | | | |
| AVRT | Age | −0.380 | <.001^{a} | −0.256 | <.001^{a} |
| | Systolic blood | −0.204 | 0.079 | −0.134 | 0.034^{a} |
| | pressure | | | | |
| | Diastolic blood | −0.023 | 0.846 | 0.001 | 0.993 |
| | pressure | | | | |
| | Intra-ocular | 0.033 | 0.781 | 0.055 | 0.390 |
| | pressure | | | | |
| | Visual field mean | NA | NA | 0.319 | <.001^{a} |
| | deviation | | | | |
| | OCT signal | 0.423 | <.001^{a} | 0.285 | <.001^{a} |
| | strength | | | | |
| TLVA | Age | −0.128 | 0.274 | −0.017 | 0.791 |
| | Systolic blood | −0.074 | 0.526 | −0.078 | 0.220 |
| | pressure | | | | |
| | Diastolic blood | 0.034 | 0.774 | −0.050 | 0.433 |
| | pressure | | | | |
| | Intra-ocular | −0.161 | 0.169 | −0.094 | 0.138 |
| | pressure | | | | |
| | Visual field mean | NA | NA | 0.188 | 0.003^{a} |
| | deviation | | | | |
| | OCT signal | 0.279 | 0.015^{a} | 0.003 | 0.957 |
| | strength | | | | |
| TCA | Age | 0.394 | <.001^{a} | −0.004 | 0.956 |
| | Systolic blood | 0.277 | 0.016^{a} | 0.066 | 0.299 |
| | pressure | | | | |
| | Diastolic blood | 0.027 | 0.820 | −0.012 | 0.846 |
| | pressure | | | | |
| | Intra-ocular | 0.078 | 0.509 | 0.136 | 0.032^{a} |
| | pressure | | | | |
| | Visual field mean | NA | NA | 0.003 | 0.967 |
| | deviation | | | | |
| | OCT signal | −0.289 | 0.012^{a} | 0.015 | 0.818 |
| | strength | | | | |
| TVA | Age | 0.217 | 0.061 | −0.020 | 0.748 |
| | Systolic blood | 0.167 | 0.153 | −0.043 | 0.499 |
| | pressure | | | | |
| | Diastolic blood | 0.054 | 0.647 | −0.062 | 0.333 |
| | pressure | | | | |
| | Intra-ocular | −0.082 | 0.482 | −0.017 | 0.792 |
| | pressure | | | | |
| | Visual field mean | NA | NA | 0.204 | 0.001^{a} |
| | deviation | | | | |
| | OCT signal | 0.012 | 0.919 | 0.013 | 0.840 |
| | strength | | | | |

Abbreviation: RNFLT, RNFL thickness; LVRT, large vessels-removed RNFL thickness; AVRT, all vessels-removed RNFL thickness; TLVA, total large vascular area; TCA, total capillaries area; TVA, total vascular area; NA, not applicable.
^{a}Statistically significant correlation (P < .05).

FIG. 12 illustrates the ROC curves for various RNFL thicknesses at different levels of vessel removal in distinguishing between normal and glaucomatous eyes. The AUCs for RNFLT and LVRT were similar (AUC: 0.91 [95% CI, 0.86-0.94] and AUC: 0.92 [95% CI, 0.87-0.95]) and there was no difference between RNFLT and LVRT in AUCs (P=0.263). In contrast, AVRT had a higher diagnostic performance (AUC: 0.94 [95% CI, 0.90-0.96]) compared to RNFLT and LVRT. There were significant differences in AUCs between AVRT and RNFLT (P<0.001), and between AVRT and LVRT (P<0.001). To evaluate the influence of large vessels and capillaries in discriminating between normal eyes and eyes with glaucoma, the study added TLVA and TCA to AVRT in a logistic regression analysis. There was no significant improvement (P>0.05) in performance when either TLVA (AUC: 0.94 [95% CI, 0.90-0.96]) or TCA (AUC: 0.95 [95% CI 0.91-0.97]) were individually used with AVRT. Instead, adding both TLVA and TCA to AVRT in the regression model significantly (P=0.027) improved the diagnostic performance (AUC: 0.95 [95% CI 0.92-0.97]).

Study Analysis

The study evaluated the diagnostic potential of the peripapillary RNFL thickness measurement with different levels of vessels exclusion in both healthy and glaucomatous eyes, using OCT and OCT-A. The study first assessed the correlations between biometric variables and our computed metrics in healthy and glaucomatous eyes. After removing the vascular component from the peripapillary RNFL, the neuronal component of the measured RNFL thickness was significantly correlated with increasing age in healthy eyes (r=−0.38, P<0.001). This is supported by the work of Chua et al. ‘ Compensation of retinal nerve fibre layer thickness as assessed using optical coherence tomography based on anatomical confounders. Br J Ophthalmol. 2020; 104(2):282-290’ and Patel et al. ‘Retinal nerve fiber layer assessment: area versus thickness measurements from elliptical scans centered on the optic nerve. Investigative ophthalmology & visual science. 2011; 52(5):2477-2489’ where the effect of aging on RNFL thickness without large vessels was shown using OCT. The study demonstrated that in glaucomatous eyes, the measured peripapillary RNFL thickness including vascular component had the highest negative correlation with age (r=−0.256, P<0.001). A possible reason is that both blood vessels and RGCs have direct contributions to peripapillary RNFL thickness. Hood et al. ‘Blood vessel contributions to retinal nerve fiber layer thickness profiles measured with optical coherence tomography. J Glaucoma. 2008; 17(7):519-528’ showed that the change in large vessel size is relatively small, but the contribution of blood vessels can make a significant contribution in glaucoma patients and lead to inaccurate assessment of functional RGCs. The study is also in agreement with this work, showing the total large vascular area is significantly correlated with the loss of visual field. In addition, RGCs loss could be due to both ageing and glaucoma progression. Hence, it is important to do the correction for age-related decline during thickness measurement when analysing the changes in peripapillary RNFL thickness due to glaucoma.

The logistic regression analysis revealed that the vascular component, especially the capillaries, plays a significant role in diagnostic performance. The results showed that the neuronal component of the measured RNFL thickness has a better diagnostic performance of AUC 0.94. The diagnostic accuracy significantly improved to AUC 0.95 (P<0.05) when the total area of the large vessels and the capillaries area were included with the neuronal component of the measured RNFL thickness in the regression analysis. This clearly demonstrated the effect of the vascular component on the discriminative ability of peripapillary RNFL thickness measurement in glaucoma and suggests that the vascular component should be considered independently from the neuronal component.

The peripapillary RNFL mainly comprises of large blood vessels, capillaries and RGCs. The neuronal component consists of RGCs and neuroglia which can be visualised using OCT. Large blood vessels could also be clearly observed in OCT scans but capillaries are not as prominent. Most of the previous works used OCT to study the contribution of large vessels in peripapillary RNFL thickness measurement. Patel et al. ‘Retinal nerve fiber layer assessment: area versus thickness measurements from elliptical scans centered on the optic nerve’ showed that the vessel contribution to RNFL thickness was greatest in the superior and inferior regions of non-human primate eyes and demonstrated the removal of major vessels can lead to a better measure for the neuronal component of the RNFL. In a study of 180 human subjects using OCT, Ye et al. ‘Impact of segmentation errors and retinal blood vessels on retinal nerve fibre layer measurements using spectral-domain optical coherence tomography’ presented a work on the impact of large vessels on RNFL thickness measurements and showed that the reliability of RNFL thickness measurement can be affected by these vessels especially in severe glaucoma when RNFL is thinned. However, peripapillary capillaries were not excluded from RNFL in these works and its contribution was not evaluated.

As a functional extension of OCT, OCT-A provides information on vascular flow in retina as well as choroid, without the need to perform intravenous dye injection. OCT-A has been widely used to study the perfusion of peripapillary capillaries in glaucomatous eyes. Among earlier works, Richter et al. ‘Peripapillary microvasculature in the retinal nerve fiber layer in glaucoma by optical coherence tomography angiography: focal structural and functional correlations and diagnostic performance Clin Ophthalmol. 2018; 12:2285-2296’ demonstrated the diagnostic performance for peripapillary vessel parameters using OCT-A enface images and showed that they outperformed the vessel parameters computed in macular region. Rao et al. ‘Diagnostic ability of peripapillary vessel density measurements of optical coherence tomography angiography in primary open-angle and angle-closure glaucoma. British Journal of Ophthalmology. 2017; 101(8):1066-1070’ also evaluated the diagnostic performance of peripapillary vessel density measurements in various peripapillary sectors of glaucomatous eyes and found that the performance was good especially in inferotemporal sector. The vessel density measurements were also found to be comparable with the RNFL thickness measurements. However, the vessel density measurement was based on en face OCT-A imaging protocol whereas, the RNFL thickness measurement was based on peripapillary circular OCT scan protocol. The current study used the ocular perfusion information provided by OCT-A to identify the peripapillary vessels for better segregation of the vascular component and neuronal component in peripapillary RNFL.

The study also evaluated the correlation between the computed metrics and IOP. There is no association found between the IOP and most of the computed metrics including RNFLT. This could be attributed to ongoing treatments to control IOP in glaucoma subjects. The study further showed that reduced visual field mean deviation was significantly associated with decreased peripapillary RNFL thickness with the vascular component removed.

There are several limitations to the study. First, the extraction of different levels of vessels is dependent on the quality of OCT and OCT-A images. Poor image quality which is caused by motion and media opacity is common and can affect the visibility of vessels. Hence, image quality checks are essential to ensure the quality of OCT and OCT-A images before performing segregation. The severity profile of glaucomatous eyes was limited by the number of severe cases. Most of the participants in this study had visual field mean deviation of better than −6 dB and thus, further evaluation on more severe glaucomatous eyes is needed. As the neuronal component also consists of neuroglia which should not be considered as part of RGCs loss, it is worth noting that this may affect the thickness measurements. Lastly, due to the cross-sectional nature of this study, causal relationships between vascular and neuronal components in glaucomatous eyes could not be evaluated.

The study evaluated the diagnostic performance of vascular and neuronal components in the peripapillary RNFL in glaucomatous eyes based on thickness measurement and vascular area. We found that the peripapillary RNFL thickness measured without vascular component performed better than when with a vascular component, in discriminating healthy and glaucomatous eyes. The diagnostic performance can be improved by including the contribution of the vascular area. Separation of neuronal and vascular components allows better appreciation of changes in neuronal components due to age or diseases. The vascular component was also associated with visual field loss and should be considered in the diagnostic evaluation of glaucoma.

Many modifications will be apparent to those skilled in the art without departing from the scope of the present invention.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A system for detecting vasculature in OCT image data of a tissue of a subject, the system comprising:
at least one processor (processors(s));
a memory accessible to the processor, the memory comprising program code executable by the processors(s) to:
receive OCT image data comprising optical coherence tomography (OCT) scan data and OCT angiography (OCTA) scan data;
segment the OCT scan data to locate a layer of interest in the tissue;
generate an en face vascular network map from the OCTA scan data;
project one or more vascular regions from the en face vascular network map onto the layer of interest in a cross-sectional image of the OCT scan data to define one or more regions of interest (ROIs), wherein respective ROIs are defined by the intersection between the vascular regions and the layer of interest; and
identify vascular objects in the one or more ROIs.

2. The system of claim 1, wherein the vascular objects are identified by: shape fitting within the ROI; a Hough transform; or a Watershed transform.

3. The system of claim 1, wherein the processor(s) is further configured to remove the vascular objects from the layer of interest to generate an image of one or more non-vascular components of the layer of interest.

4. The system of claim 1, wherein the processor(s) is further configured to determine one or more clinical parameters based on the identified vascular objects and/or the image of the one or more non-vascular components.

5. The system of claim 1, wherein the tissue is a retina of the subject and the one or more vascular regions in the en face vascular map reside in a circumpapillary region.

6. The system of claim 4, wherein the one or more clinical parameters comprise circumpapillary retinal nerve fibre layer (RNFL) thickness.

7. The system of claim 1, wherein processor(s) is further configured to select the layer of interest according to a disease model.

8. A method of detecting vasculature in OCT image data of a tissue of a subject, the OCT image data comprising optical coherence tomography (OCT) scan data and OCT angiography (OCTA) scan data, the method comprising:
segmenting the OCT scan data to locate a layer of interest in the tissue;
generating an en face vascular network map from the OCTA scan data;
projecting one or more vascular regions from the en face vascular network map onto the layer of interest in a cross-sectional image of the OCT scan data to define one or more regions of interest (ROIs), wherein respective ROIs are defined by the intersection between the vascular regions and the layer of interest; and
identifying vascular objects in the one or more ROIs.

9. A method according to claim 8, wherein the tissue is a retina of the subject.

10. A method according to claim 8, wherein the vascular objects are identified by: shape fitting within the ROI; a Hough transform; or a Watershed transform.

11. A method according to claim 8, comprising removing the vascular objects from the layer of interest to generate an image of one or more non-vascular components of the layer of interest.

12. A method according to claim 11, wherein the one or more non-vascular components comprise a neuronal component.

13. A method according to claim 8, wherein said segmenting is carried out using a convolutional neural network.

14. A method according to claim 12, wherein the convolutional neural network is U-Net.

15. A method according to claim 8, comprising determining one or more clinical parameters based on the identified vascular objects and/or the image of the one or more non-vascular components.

16. A method according to claim 9, wherein the one or more vascular regions in the en face vascular map reside in a circumpapillary region.

17. A method according to claim 15, wherein the one or more clinical parameters comprise circumpapillary retinal nerve fibre layer (RNFL) thickness.

18. A method according to claim 8, wherein the layer of interest is selected according to a disease model.

19. A system for detecting vasculature in OCT image data of a tissue of a subject, the OCT image data comprising optical coherence tomography (OCT) scan data and OCT angiography (OCTA) scan data, the system comprising at least one processor in communication with machine-readable storage having stored thereon instructions for causing the at least one processor to carry out a method according to claim 8.

20. Non-transitory computer-readable storage having stored thereon processor-executable instructions for causing at least one processor to carry out a method according to claim 8.
